(19) United States
(12) Patent Application Publication
(10) Pub. No.: US 2008/0069780 A1
(43) Pub. Date: Mar. 20, 2008

(54) High Concentration Single Phase Gycol Aerosol Air Sanitizer with Dimethyl Ether Propellant/Solvent

(75) Inventors: Carl S. Weiss, Buffalo Grove, IL (US); Cary E. Manderfield, Racine, WI (US); John A. Schroeder, Racine, WI (US)

Correspondence Address: S.C. JOHNSON & SON, INC., 1525 HOWE STREET, RACINE, WI, 53403-2236, US

(73) Assignee: S.C. JOHNSON & SON, INC., Racine, WI (US)

(21) Appl. No.: 11/931,045

(22) Filed: Oct. 31, 2007

Related U.S. Application Data
(63) Continuation-in-part of application No. 11/696,233, filed on Apr. 4, 2007.
(60) Provisional application No. 60/744,298, filed on Apr. 5, 2006.

(51) Int. Cl. A61K 9/12; A61P 31/00

(52) U.S. Cl. 424/45

(57) ABSTRACT

Improved single-phase aerosol spray air sanitizers and deodorizers are disclosed. The preferred active ingredient for the air sanitization is a high concentration of triethylene glycol (TEG), in a single-phase aerosol formulation made possible by the use of dimethyl ether (DME) as a propellant. The combination of TEG and DME enables high concentrations of TEG to be utilized in a single-phase aerosol formulation, without requiring the use of ethanol or water as co-solvents. The formulation is useful for sanitizing air and removing bacteria and other malodorants from the air as well achieving higher bacteria kill rates than existing air sanitization formulations due to the high concentrations of TEG. Because the high concentration TEG aerosol formulation is a single-phase formulation, it can be utilized in automated spray or dispensing systems and do not require shaking or agitation prior to use. Disclosed formulations leave little or no visible residue on hard surfaces when dispensed into the air of a room.

### CROSS REFERENCE TO RELATED APPLICATIONS

This is a continuation-in-part of application Ser. No. 11/696,233, filed on Apr. 4, 2007 and still pending, which claims priority to Provisional Application Ser. No. 60/744,298, filed on Apr. 5, 2006.

### BACKGROUND

1. Technical Field

Air sanitizers/deodorants are provided with novel combinations of glycol and dimethyl ether propellant that enable high concentrations of glycol to be utilized in single phase aerosol formulations, but do not leave a substantial residue if used over a hard or soft surface. Methods for sanitizing air and/or mitigating airborne malodor using high concentrations of glycol in single phase aerosol formulations are also disclosed.

2. Description of the Related Art

A wide variety of deodorizing compositions are known in the art, the most common of which contain perfumes or fragrances to mask malodor. Odor masking is the intentional concealment of one odor by the addition of another. The masking of odors is typically accomplished by using perfumes or fragrances. However, high levels of fragrance are needed to ensure that the malodor is no longer noticeable or suitably masked and the masking techniques do nothing to remove or modify the source of the odor.

Malodorant modification, where the malodorant is changed, e.g., by chemical modification, has also been used. Current malodorant modification methods include oxidative degradation and reductive degradation. Oxidative degradation employs oxidizing agents such as oxygen bleaches, chlorine, chlorinated materials such as sodium hypochlorite or chlorine dioxide, and potassium permanganate to reduce malodors. Reductive degradation strategies employ reducing agents such as sodium bisulfite to reduce malodors. Most oxidation and reduction strategies are unacceptable for general household air sanitization applications due to toxicity and biocompatibility issues associated with the oxidizing/reducing agents.

Aerosol spray air sanitizers are known in the art to effectively kill airborne microorganisms and mitigate airborne malodorants. The active ingredients in currently available aerosol air sanitizers vary. One family of products relies upon glycols such as propylene glycol, dipropylene glycol and triethylene glycol. The most popular is triethylene glycol, the active ingredient in the OUST® family of products sold by S. C. Johnson & Son, Inc., the assignee of this application. Triethylene glycol (TEG) is known to kill certain airborne bacteria. TEG is also safe for use in aerosol sprays. The structure of TEG is as follows: 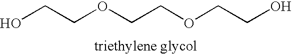
Because the solubility of TEG in conventional hydrocarbon propellants is generally low, additional solvents, such as ethanol, are added to TEG-based aerosol compositions to increase the solubility of TEG in hydrocarbon propellants. Ethanol is also used as co-solvent to increase volatility of the product and to make other components such as fragrance oils more soluble.

However, the use of ethanol and other conventional solvents in combination with conventional short C-chain aliphatic propellants limits the amount of TEG that can be included in a single-phase formulation. For example, the concentration of TEG in a single-phase aerosol composition using conventional hydrocarbon propellant is limited to no more than 6-8 wt % with 30 wt % propellant and the rest being ethanol. The use of TEG in amounts that exceed 12 or 15% normally results in a two-phase system, thereby requiring the consumer to vigorously shake the canister before use and eliminating use of the formulation in a continuous or automated spray device. Single-phase aerosol products that do not require vigorous shaking are advantageous because consumers often forget to shake multi-phase products that require mixing and, because the performance of single-phase aerosol products is not dependent upon mixing, the performance of single-phase aerosols can be more consistent. In contrast, the efficacy of multi-phase aerosol products that require mixing can be somewhat inconsistent, especially if the user is physically unable to shake the can as vigorously as required.

An additional problem associated with aerosol air fresheners and/or air sanitizers is that they can leave a visible residue on surfaces such as furniture and countertops disposed within the room in which the product is used. Obviously, such visible residues are unsightly and not preferred by consumers. Thus, it is desirable to provide aerosol air sanitizers and/or air fresheners that are substantially residue-free. The combination of a single-phase and substantially residue-free air sanitization product would also be highly desirable.

The use of the terms “sanitizing” and “disinfecting” herein is consistent with Environmental Protection Agency Disinfectant Technical Science Section (DIS-TSS) nos. 01, 08, 11 and 13 (http://www.epa.gov/oppad001/sciencepolicy.htm).

For example, in regard to hard surface cleaning products, DIS-TSS-01 requires a product labeled as a “disinfectant” to be tested with sixty carriers, each with three different samples (for a total of 180 samples), representing three different batches, one of which is at least 60 days old, against Salmonella choleraesuis (ATCC 10708—Gram negative) or Staphylococcus aureus (ATCC 6538—Gram positive). Under DIS-TSS-01, to support a label claim of the product being a “disinfectant,” the product must provide a complete kill 59 of 60 carriers at a 95% confidence level. Thus, under DIS-TSS-01, a complete kill is essentially required for label claims of effectiveness as a “general disinfectant” or representations that the product is effective against a broad spectrum of microorganisms, including Gram-positive and Gram-negative bacteria.

In contrast to “disinfecting,” which refers to a complete kill of all bacteria on a test (hard) surface, the term “sanitizing” refers to a less than complete kill of the bacteria in air or on a soft surface. Because experimental data is available to show that air sanitizers designed for household use do not sterilize, disinfect, act as a germicide, or protect experimental animals from infections by airborne bacteria or viruses, EPA regulations currently prohibit label claims of “disinfectant” on products used in air or on soft surfaces. In fact, the EPA imposes separate requirements for the label use of “sanitizing” for air (DIS-TSS-11) and for “sanitizing” certain soft surfaces like carpeting (DIS-TSS-08).

DIS/TSS-11 applies to products with label claims of reducing airborne microorganisms or bacteria. Glycol vapors have been shown to produce significant decreases in numbers of viable airborne bacteria within enclosed spaces. Aerosol formulations including glycols (triethylene, dipropylene, or propylene glycol) at concentrations of 5% or more will temporarily reduce numbers of airborne bacteria when adequate amounts are dispensed within a room. Unlike DIS-TSS-01, no standard method for evaluating air sanitizers has been adopted and incorporated into DIS-TSS-11.

Existing commercialize products claiming to sanitize air typically have a glycol concentration ranging from 6% to 9%, with some regional products regional level as high as 12%. The conventional propellants for these aerosol products are typically propane, butane, isobutene, or a mixture thereof. With those propellants, a higher concentration of glycol often results in a two-phase or even three-phase system. Therefore, the application of an aerosol composition that contains a high concentration of glycol and conventional propellants requires extensive shaking before dispensing into the air, which is both time consuming and tedious to a consumer.

While most currently available products satisfy the requirements of DIS-TSS-1, a higher level of microbial efficacy is desired. Specifically, airborne bacteria can be dangerous and, under certain conditions, it is desirable to achieve a higher kill rate than currently-available air sanitization products. Further, TEG and other glycols are known to be capable of removing malodorant molecules from the air and it would be advantageous to exploit such a mechanism while achieving a level of air sanitization.

Both ethanol and hydrocarbon propellants are considered to be Volatile Organic Compounds (VOCs). The content of VOCs in aerosol air sanitizers has the potential to be regulated by federal and/or state regulatory agencies, such as the Environmental Protection Agency (EPA) and California Air Resource Board (CARB). An existing single-phase TEG-based aerosol composition typically has a VOC content of more than 90 wt %. By increasing the content of TEG, the VOC content of the aerosol composition can be reduced as well.

Despite all of the above efforts to develop methods for controlling airborne microorganisms and mitigating airborne malodors, there is still a need for an improved method to expedite the effective control of airborne microorganisms and malodors, Further, there is still a need for an improved air sanitizer/deodorant that contains a high concentration of glycol to significantly increase the antimicrobial and deodorant efficacy thereof while maintaining the formulation in a single-phase. Finally, there is still a need to decrease the VOC content of single-phase glycol-based aerosol compositions and providing such single-phase glycol-based aerosol compositions that are substantially residue-free or that do not leave a substantial residue on surfaces disposed within a room in which the product is used for air sanitization, air deodorizing and/or air freshening.

### SUMMARY OF THE DISCLOSURE

An improved formulation for sanitizing air by killing airborne bacteria is provided. The formulation may be provided in an aerosol spray form. The aerosol formulation provides a high concentration of an active ingredient for air sanitization in a single-phase aerosol formulation that has been previously unavailable. By providing an aerosol formulation in a single-phase, the need to shake the container prior to use is eliminated. “Single-phase,” as used herein, means the liquid formulation is homogeneous and substantially free of phase separation.

In at least one disclosed embodiment, the formulation is both single-phase and at least substantially residue-free, meaning that the product will not leave substantial amounts of visible residue on nearby hard surfaces after the product is sprayed in the air of a room.

In a refinement, the active ingredient for air sanitization is a glycol. In an embodiment, the active ingredient for air sanitization is selected from the group consisting of triethylene glycol (TEG), dipropylene glycol, propylene glycol, and mixtures thereof. In a preferred embodiment, the active ingredient is TEG. Preferably, the active ingredient for air sanitization is present in higher concentrations than currently employed in single phase aerosol formulations. Other glycols that are capable of sanitizing air will be apparent to one of ordinary skill in the art.

According to one embodiment, the propellant used in the aerosol formulation is an ether propellant. In a refinement, the preferred propellant is dimethyl ether (DME; CH₃—O—CH₃). Other ethers including, but are not limited to, methyl ethyl ether, fluorinated dimethyl ether, and fluorinated methyl ethyl ether, may also be used as propellant as an substitute to, or in conjunction with, DME.

It is unexpected and surprising that the use of the ether propellant enables the inclusion of glycol in the aerosol formulation at a concentration substantially higher than what is currently available in a single-phase aerosol formulation that includes the glycol as the active ingredient. As a result, the amount of the active ingredient for air sanitization delivered into the air may be significantly increased without the drawbacks of a multi-phase aerosol formulation that requires shaking before each application. Therefore, the air sanitizing performance of the aerosol formulation may be significantly improved without sacrificing the convenience provided by the single-phase product. Without being bound by any particular theory, it is contemplated that, in addition to propelling the formulation into the air in a form of aerosol, the ether propellant also functions as a solvent or co-solvent for the active ingredient for air sanitization as well as other ingredients of the aerosol formulation thereby making additional co-solvents such as water or an alcohol optional.

The aerosol formulation may include a co-propellant. The co-propellant may be any conventional propellant that is compatible with other ingredients of the aerosol formulation. Preferably, the inclusion of the co-propellant does not affect the single-phase presence of the aerosol formulation. Suitable co-propellants include hydrocarbons, halogen-substituted hydrocarbons, carbon dioxide, compressed air, compressed nitrogen, etc. In one refinement, the co-propellant is a B-52 propellant, which is a mixture of butane and propane. Other co-propellants may be included that will be apparent to those skilled in the art.

One or more co-solvents in addition to DME may also be included in the aerosol formulation. Preferably, the inclusion of a co-solvent should not affect the single-phase presence of the aerosol formulation. The co-solvent can be water or one or more alcohols or a mixture thereof. In a refinement, when water is used as a co-solvent, another co-solvent may be used in the form of a monohydric alcohol, preferably a short chain monohydric alcohol such as ethanol. One co-solvent a mixture of water and ethanol. Isopropanol, butanol and propanol can also be used as co-solvents with DME. Thus, the co-solvent may be selected from the group consisting of water, ethanol, isopropanol, butanol, propanol, and mixtures thereof.

However, in at least one embodiment, a co-solvent is not utilized and the product is a mixture of DME, TEG and fragrance.

A wide range of glycol concentrations may be used. In one embodiment, the single-phase aerosol formulation comprises no less than about 15 wt % glycol as higher amounts of glycol are preferred for purposes of exploiting the ability of glycols to sanitize air or kill airborne bacteria. In a refinement, the single-phase aerosol formulation comprises no less than about 20 wt % glycol. In another refinement, the single-phase aerosol formulation comprises no less than about 25 wt % glycol. In yet another refinement, the single-phase aerosol formulation comprises no less than about 30 wt % glycol. In another embodiment, the single-phase aerosol formulation comprises about 31 wt % glycol, which is most preferably TEG. It is contemplated that the inclusion of glycol at other appropriate concentrations will be apparent to those of ordinary skill in the art. For example, lower amounts of glycol (e.g. less than 15 wt %) may be satisfactory for some applications.

Similarly, the ether propellant may be present in a wide range of concentrations. In an embodiment, the single-phase aerosol formulation comprises from about 10 to about 85 wt % ether propellant. In a refinement, the single-phase aerosol formulation comprises from about 25 to about 80 wt % ether propellant. In a further refinement, the single-phase aerosol formulation comprises from about 40 to about 85 wt % ether propellant. In another embodiment, the single-phase aerosol formulation comprises from about 55 to about 75 wt % ether propellant and more preferably about 65 wt % ether propellant, which is most preferably DME. The inclusion of ether propellant at other appropriate concentrations will be apparent to those skilled in the art.

Indeed, one advantage of a single-phase, alcohol-free, water-free formulation that includes DME, TEG and fragrance is the lack of any need for a corrosion inhibitor and the added expense associated with using a corrosion inhibitor.

According to another refinement, the formulation may also comprise one or more perfumes or fragrances. Preferably, the inclusion of the fragrance does not affect the single-phase presence of the aerosol formulation. An alcohol may serve as a solvent to the one or more fragrances as it enhances the solubility of most commercially available fragrances used for aerosol sprays, which are typically hydrocarbons. The ether propellant may serve this function as well. The fragrance content can vary widely, depending upon the specific application. Specifically, the fragrance can be present in an amount ranging from about 0.01 up to about 6 wt %. Many applications will only require from about 0.01 to about 0.5 wt %. However, specific malodors associated with bathrooms, mold/mildew, pet urine such as cat urine and smoke may require higher fragrance amounts ranging from about 1 to about 4 wt %. For a single-phase formulation used in an automatic dispensing device, a fragrance amount ranging from about 2 to about 5.6 wt % will typically be sufficient for a 60 μl metered dose (˜50 mg).

One disclosed aerosol formulation comprises an ether propellant and a glycol selected from the group consisting of triethylene glycol, dipropylene glycol, propylene glycol, and mixtures thereof, wherein the aerosol formulation has a single-phase presence at room temperature. Moreover, compared to existing single-phase TEG-based aerosol compositions, some embodiments of the present disclosure can reduce the VOC content of the composition to 70 wt % or lower.

Broadly, disclosed aerosol formulations can comprise from about 15 to about 75 wt % glycol, from about 20 to about 75 wt % ether propellant, and optionally, one or more fragrances.

In another refinement, the formulation comprises from about 25 to about 35 wt % glycol and about from about 55 to about 75 wt % ether propellant. In another refinement, the formulation comprises about 30 wt % glycol and about 65 wt % ether propellant. In still another refinement, the formulation comprises about 31 wt % glycol and 65 wt % ether propellant. In still another refinement, the formulation comprises about 65 wt % ether propellant, about 31.5 wt % glycol, and about 3.5 wt % fragrance.

In yet another refinement, if water is used as a co-solvent, the aerosol formulation contains no more than about 29 wt % water.

It will be noted that the above formulations are single-phase formulations and therefore does not require shaking or mixing prior to use. Therefore, the above formulations can be utilized in an automated system that dispenses the formulation continuously, periodically or at timed intervals. Because one advantage of the disclosed formulations is that they do not leave a visible residue on nearby surfaces, they are ideally suited for both automated dispensing devices as well as conventional aerosol dispensers used manually.

A method for sanitizing air and removing malodorant molecules from air comprises providing the single-phase aerosol formulation described above, spraying the formulation in the air, and allowing the atomized formulation to interact with airborne bacteria and engage airborne malodorant molecules.

A method for automatically and/or periodically dispensing a high concentration glycol solution comprises providing the single-phase aerosol formulation described above and either continuously or periodically dispensing predetermined amounts of the formulation into the air or ambient environment of an enclosed room or living space.

A method for automatically and/or periodically treating malodors in an enclosed room or living space comprises providing a single-phase aerosol formulation comprising a high concentration of glycol and ether propellant as described above and either continuously or periodically dispensing predetermined amounts of the formulation into the air of the enclosed room or living space.

Other advantages and features of the disclosed embodiments and methods will be described in the following detailed description of the presently preferred embodiments.

### DETAILED DESCRIPTION OF THE PRESENTLY PREFERRED EMBODIMENTS

According to one embodiment, a single phase aerosol formulation comprising: an ether propellant selected from the group consisting of dimethyl ether, methyl ethyl ether, fluorinated dimethyl ether, and fluorinated methyl ethyl ether; and a glycol selected from the group consisting of triethylene glycol (TEG), dipropylene glycol, propylene glycol, and mixtures thereof may be used to deliver a high concentration of glycol to the air in a form of an aerosol spray for sanitization and/or deodorizing purposes. The aerosol formulation may optionally include a co-solvent, a co-propellant, a fragrance, and one or more corrosion inhibitors. The aerosol formulation preferably has a single-phase presence at room temperature so that no shaking or mixing is required prior to application. By “single-phase” it is meant that the liquid formulation is homogeneous and substantially free of phase separation.

Glycol

The preferred active ingredient for air sanitization is one or more glycols. It has been known to use certain glycols in aerosols or vapor forms to sanitize or disinfect the air in a room by killing airborne bacteria that are often a source of odors. Among those glycols, triethylene glycol (TEG) has been found particularly effective for sanitizing air when delivered via an aerosol spray. The commercially successful OUST® air sanitizer products utilize a mixture that contains about 6 wt % TEG.

TEG is a colorless, odorless, non-volatile and hygroscopic liquid. It is characterized by two hydroxyl groups along with two ether linkages which contribute to its high water solubility, hygroscopicity and its ability to neutralize airborne odor-causing bacteria in the air. TEG can be prepared commercially by the oxidation of ethylene at high temperatures in the presence of a silver oxide catalyst, following by hydration of the ethylene oxidate to yield mono-, di-, tri- and tetra-ethylene glycol products. Moreover, TEG has a low toxicity, as compared to some other glycols such as diethylene glycol (DEG).

Other glycols, such as dipropylene glycol and propylene glycol, may also be use as a substitute to, or in conjunction with, TEG.

In contrast to existing commercialize products claiming to sanitize air typically have a glycol concentration ranging from 6% to 9%, with some regional products regional level as high as 12%, the aerosol formulation of one embodiment comprises no less than about 15 wt % glycol. In other embodiments, the single-phase aerosol formulation comprises from about 15 to in excess of 30 wt % glycol. As shown in Examples 1-16 below, a wide range of glycol concentrations may be accommodated within the scope of this disclosure. Further, the inclusion of glycol at other appropriate concentrations will be apparent to those of ordinary skill in the art.

Ether Propellant

Aerosol propellant is an essential element in any aerosol formulation as it provides constant pressure for dispensing the formulation through the nozzle. For commercial aerosols, the propellant or propellant mixture will typically have a boiling point slightly lower than room temperature. As a result, inside the pressurized can, the vapor phase of the propellant exists in equilibrium with the liquid phase of the propellant at a vapor pressure that is higher than atmospheric pressure, and as a result, the vapor phase of the propellant is able to drive the formulation out of the can when the nozzle is opened. Moreover, as the vapor phase of the propellant escapes through the nozzle, it is immediately replenished by evaporation of the liquid phase of the propellant as equilibrium within the can is reestablished.

One preferred propellant according to this disclosure is an ether propellant, preferably DME. Optionally, a co-propellant such as one or more hydrocarbons, halogen-substituted hydrocarbons, carbon dioxide, compressed air, compressed nitrogen, etc, may also be included.

The ether propellant may be present in the single-phase aerosol formulation in a wide range of concentrations. The aerosol formulation may comprise from about 10 to about 85 wt % ether propellant. In one embodiment, the single-phase aerosol formulation comprises from about 25 to about 75 wt % ether propellant. As shown in Examples 1-16 below, a wide range of propellant concentrations may be used and still fall within the scope of this disclosure. Further, the inclusion of the ether propellant at other appropriate concentrations will be apparent to those of ordinary skill in the art.

Co-Solvent

While the liquefied DME serves as a solvent to improve the solubilization of the glycol and other ingredients of the aerosol formulation, a co-solvent may also be included to further facilitate solubilization. Preferably, the co-solvent is selected from the group consisting of water, low molecular monohydric alcohols, and mixtures thereof.

One suitable co-solvent is water due to its low cost and availability. Preferably, water is present in the aerosol formulation in an amount of less than about 50 wt %. In an embodiment, water is present in the aerosol formulation of less than about 15 wt %.

Another suitable co-solvent is a low molecular weight monohydric alcohol, such as ethanol, propanol, isopropanol, and butanol. Preferably, the alcohol is present in the aerosol formulation in an amount ranging from about 0 to less than about 40 wt %. According to one refinement, the preferred alcohol co-solvent is ethanol. Preferably, the total content of the co-solvents is less than about 70 wt %.

As shown in Examples 1-16 below, a wide range of co-solvent concentrations may be accommodated within the scope of this disclosure. Further, the inclusion of the co-solvent at other appropriate concentrations will be apparent to one of ordinary skill in the art.

Corrosion Inhibitor

The introduction of DME as an aerosol propellant has opened the way to the use of more water-based aerosol formulations and made possible the manufacture of products of lesser flammability and lower ingredient cost. However, the use of water in such aerosol formulations also increases the problem of corrosion on the interior of metallic cans, thus leading to contamination of the aerosol product and ultimately to leaking of the can if the corrosion is severe enough. For this reason, corrosion inhibitors are preferably used with aerosol propellants containing DME, when this propellant is to be used in metallic cans containing a water-based formulation.

If a canister susceptible to corrosion is employed with a formulation containing water, one or more corrosion inhibitors may be included such as potassium phosphates, potassium nitrite, sodium phosphates, sodium nitrite, mixtures thereof, or one or more other corrosion inhibiting agents as shown in Examples 2-7 below.

Di-potassium phosphate (K₂HPO₄) is useful as both a corrosion inhibitor and a buffer. Di-potassium phosphate may be used alone or in combination with mono-potassium phosphate (KH₂PO₄). Di-sodium phosphate (Na₂HPO₄) is also useful as both a corrosion inhibitor and a buffer and may be substituted for the di-potassium phosphate. Mono-sodium phosphate (NaH₂PO₄) may also be used instead of or in addition to mono-potassium phosphate. The combination of di alone or di and mono-potassium and/or sodium phosphates has been found to be enhanced by the presence of another corrosion inhibitor in the form of potassium nitrite (KNO₂) and/or sodium nitrite (NaNO₂). Accordingly, the presence of di-potassium phosphate or di-sodium phosphate may range from about 0.01 to about 1.0 wt %, more preferably from about 0.02 to about 0.25 wt %. A suitable pH range for these salts is from about 7 to about 11, with a preferred range from about 8 to about 10.

The amount of di-potassium phosphate or di-sodium phosphate may be reduced if a small amount of mono-potassium phosphate and/or mono-sodium phosphate is utilized as shown above in Examples 2 and 4, but the use of only di- or only mono-phosphates is possible. If used, the mono-potassium phosphate and/or mono-sodium phosphate need only be present in small amounts, but their presence may range from about 0.01 to about 1.0 wt %, more preferably around about 0.02 wt %. If utilized, the potassium nitrite can be present in amount ranging from about 0.01 to about 1.0 wt %, more preferably from about 0.07 to about 0.15 wt %. Further, to achieve the same objectives, the inhibitor may also be generated in situ with potassium hydroxide and phosphoric acid or with sodium hydroxide and phosphoric acid. The mono-potassium/sodium phosphates may be added in amounts exceeding that of the di-potassium/sodium phosphates to create buffer systems ranging from acidic to alkaline pHs ranging from about 5 to about 10, preferably from about 7 to about 9.

Also, ammonium phosphates and/or ammonium nitrite may be used or combined with the corrosion inhibitors discussed above. However, ammonium nitrite is explosive and therefore presents handling problems. Tri-potassium and tri-sodium phosphates could also be used and neutralized to an acceptable pH with an acid such as phosphoric acid. Triethanolamine with sodium benzoate or with one or more the other inhibitors discussed above is a less preferred alternative for corrosion inhibition. As another alternative, corrosion inhibition may be provided by borax (Na₂B₄O₇.H₂O) alone or in combination with sodium nitrite or with one more of the other inhibitors discussed above.

Other suitable corrosion inhibitors apparent to those of ordinary skill in the art may also be included in the aerosol formulation.

Fragrance

Optionally, the aerosol formulation may comprise one or more fragrances for masking malodors and increasing elegance. As is well known, a fragrance normally consists of a mixture of a number of fragrant materials, each of which has a particular fragrance. The number of fragrant materials in a fragrance is typically ten or more. The range of fragrant materials used may vary. The materials come from a variety of chemical classes, but in general are water-insoluble oils. In many instances, the molecular weight of a fragrance material is in excess of 150, but does not exceed 300.

Fragrance oils may be obtained from a variety of sources including Quest International (recently acquired by Givaudan S.A.; www.givaudan.com) of the Netherlands, Takasago International of Japan (www.takasago.com) and Firmenich International S.A. of Geneva Switzerland (www.firmenich.com). Other fragrance suppliers are well known to those skilled in the art.

The fragrance included in the aerosol formulation may be present in an amount that is sufficient to deliver a pleasant smell that can be perceived by a consumer. In the presence of a malodor, the fragrance included in the aerosol formulation may be present in an amount that masks at least a substantial portion of the malodor in the air. More preferably, the fragrance included in the aerosol formulation is preferably present in an amount that not only completely masks the odor associated with airborne microorganisms, but also delivers a pleasant smell to be perceived by a consumer. In one embodiment, the fragrance is present in the aerosol formulation in an amount of from about 0.01 to about 5 wt %.

The amount of the fragrance that is needed to mask the odor associated with airborne microorganisms, and/or the amount of the fragrance to deliver the pleasant smell to be perceived by the consumer will be apparent to those skilled in the art. For example, certain odors, such as odors associated with bathrooms, mold/mildew, cat urine and smoke may require higher concentrations of fragrance ranging from about 0.9 to about 5.0 wt %, for example. Essentially, the fragrance load or amount will depend upon the placement or application of the dispenser and whether or not the dispenser is an automated dispenser. As the single-phase formulations disclosed herein are particularly useful for automatic metered dispensers, the amount of fragrance that should be included may very well depend upon the size of the metered dose. One automated dispenser that is anticipated for use with a formulation including DME, TEG and fragrance has a 60 μl valve and dispenses about 48 mg of formulation per dose. In such a formulation, a fragrance content of about 3.5 wt % has been found to be sufficient.

If a fragrance is utilized in the aerosol formulation, at least some ethanol or other alcohol co-solvent may be preferably included in the aerosol formulation to facilitate the solubilization of the fragrance. Without being bound by a particular theory, it is contemplated that DME and TEG may also assist in this function as well.

The fragrance may comprise one or more fragrant materials or materials that provide chemically active vapors. In one embodiment, the fragrance can comprise and/or include volatile, fragrant compounds including, but not limited to natural botanic extracts, essences, fragrance oils, synthetic fragrant materials and so forth. As is known in the art, many essential oils and other natural plant derivatives contain large percentages of highly volatile scents. In this regard, numerous essential oils, essences, and scented concentrates are commonly available from companies in the fragrance and food businesses. Exemplary oils and extracts include, but are not limited to, those derived from the following plants: almond, amyris, anise, armoise, bergamot, cabreuva, calendula, canaga, cedar, chamomile, coconut, eucalyptus, fennel, jasmine, juniper, lavender, lemon, orange, palm, peppermint, quassia, rosemary, thyme, and so forth.

Without being bound by a particular theory, it is believed that an enhanced microbial air sanitizing function is provided by the single-phase aerosol formulation disclosed herein. Another benefit of the single-phase aerosol formulation is the reduction in corrosion potential or the elimination of corrosion potential with water-free formulations. Still further, the single-phase aerosol formulation disclosed herein does not require shaking prior to application and therefore can be utilized in stationary (wall-mounted) and/or automatic systems and dispensers. Finally, VOC content can be reduced with the single-phase formulations of Examples 8-10 and 14-16 below that are alcohol-free.

### EXAMPLE 1

| wt % | Description/Function | Chemical/Trade Name |
|---|---|---|
| 20% | sanitizing agent/deodorant | triethylene glycol (TEG) |
| 49.85% | propellant/solvent | dimethyl ether (DME) |
| 0% | solvent | deionized water |
| 30% | solvent | ethanol |
| 0.15% | mixture of fragrances | TAKASAGO ™ RK 1428; IFF |
| | | 1401 HBA; Firmenich SJ |
| | | 446138; or similar |
| 100% | | |

### EXAMPLE 2

| wt % | Description/Function | Chemical/Trade Name |
|---|---|---|
| 25% | sanitizing agent/deodorant | triethylene glycol (TEG) |
| 49.65% | propellant/solvent | dimethyl ether (DME) |
| 12.5% | solvent | deionized water |
| 12.5% | solvent | ethanol |
| 0.02% | corrosion inhibitor/buffer | KH₂PO₄, mono-potassium |
| | | phosphate |
| 0.18% | corrosion inhibitor/buffer | K₂HPO₄, di-potassium |
| | | phosphate |
| 0.15% | mixture of fragrances | TAKASAGO ™ RK 1428; IFF |
| | | 1401 HBA; Firmenich SJ |
| | | 446138; or similar |
| 100% | | |

Example 2 exhibits a single-phase presence when pressurized and observed in a glass bottle. Moreover, no apparent corrosion is observed when Example 2 is pressurized in a 70 mm aluminum can.

### EXAMPLE 3

| wt % | Description/Function | Chemical/Trade Name |
|---|---|---|
| 30% | sanitizing agent/deodorant | triethylene glycol (TEG) |
| 49.5% | propellant/solvent | dimethyl ether (DME) |
| 10% | solvent | deionized water |
| 10% | solvent | ethanol |
| 0.02% | corrosion inhibitor/buffer | KH₂PO₄, mono-potassium |
| | | phosphate |
| 0.18% | corrosion inhibitor/buffer | K₂HPO₄, di-potassium |
| | | phosphate |
| 0.12% | corrosion inhibitor | KNO₂, potassium nitrite |
| 0.18% | mixture of fragrances | TAKASAGO ™ RK 1428; IFF |
| | | 1401 HBA; Firmenich SJ |
| | | 446138; or similar |
| 100% | | |

Example 3 exhibits a single-phase presence when pressurized and observed in a glass aerosol bottle. Moreover, no apparent corrosion is observed when Example 3 is pressurized in a 70 mm aluminum can. Further, when Example 3 is pressurized in an unlined zinc-plated steel can, no apparent corrosion is observed.

### EXAMPLE 4

| wt % | Description/Function | Chemical/Trade Name |
|---|---|---|
| 35% | sanitizing agent/deodorant | triethylene glycol (TEG) |
| 49.65% | propellant/solvent | dimethyl ether (DME) |
| 7.5% | solvent | deionized water |
| 7.5% | solvent | ethanol |
| 0.02% | corrosion inhibitor/buffer | NaH₂PO₄, mono-sodium |
| | | phosphate |
| 0.18% | corrosion inhibitor/buffer | Na₂HPO₄, di-sodium |
| | | phosphate |
| 0.15% | mixture of fragrances | TAKASAGO ™ RK 1428; IFF |
| | | 1401 HBA; Firmenich SJ |
| | | 446138; or similar |
| 100% | | |

### EXAMPLE 5

| wt % | Description/Function | Chemical/Trade Name |
|---|---|---|
| 40% | sanitizing agent/deodorant | triethylene glycol (TEG) |
| 49.5% | propellant/solvent | dimethyl ether (DME) |
| 2.5% | solvent | deionized water |
| 7.5% | solvent | ethanol |
| 0.02% | corrosion inhibitor/buffer | NaH₂PO₄, mono-sodium |
| | | phosphate |
| 0.18% | corrosion inhibitor/buffer | Na₂HPO₄, di-sodium |
| | | phosphate |
| 0.12% | corrosion inhibitor | NaNO₂, sodium nitrite |
| 0.18% | mixture of fragrances | TAKASAGO ™ RK 1428; IFF |
| | | 1401 HBA; Firmenich SJ |
| | | 446138; or similar |
| 100% | | |

### EXAMPLE 6

| wt % | Description | Chemical/Trade Name |
|---|---|---|
| 50% | sanitizing agent/deodorant | triethylene glycol (TEG) |
| 29.55% | propellant/solvent | dimethyl ether (DME) |
| 10% | solvent | deionized water |
| 10% | solvent | ethanol |
| 0.2% | corrosion inhibitor | Na₂B₄O₇•H₂O, Borax |
| 0.1% | corrosion inhibitor | NaNO₂, sodium nitrite |
| 0.15% | mixture of fragrances | fragrance oils |
| 100% | | |

Example 6 exhibits a single-phase presence when pressurized and observed in a glass aerosol bottle. Moreover, no apparent corrosion is observed when Example 6 is pressurized in a 70 mm aluminum can.

### EXAMPLE 7

| wt % | Description | Chemical/Trade Name |
|---|---|---|
| 70% | sanitizing agent/deodorant | triethylene glycol (TEG) |
| 25.55% | propellant/solvent | dimethyl ether (DME) |
| 4% | solvent | ethanol |
| 0.1% | corrosion inhibitor | triethanolamine |
| | | (HOCH₂CH₂)₃N |
| 0.2% | corrosion inhibitor | sodium benzoate, C₇H₅O₂Na |
| 0.15% | fragrance oils | fragrance oils |
| 100.00% | | |

Example 7 exhibits a single-phase presence when pressurized and observed in a glass aerosol bottle. Moreover, no apparent corrosion is observed when Example 7 is pressurized in a 70mm aluminum can.

### EXAMPLE 8

| wt % | Description/Function | Chemical/Trade Name |
|---|---|---|
| 50% | sanitizing agent/deodorant | triethylene glycol (TEG) |
| 49.85% | propellant/solvent | dimethyl ether (DME) |
| 0.15% | mixture of fragrances | TAKASAGO ™ RK 1428; IFF |
| | | 1401 HBA; Firmenich SJ |
| | | 446138; or similar |
| 100% | | |

### EXAMPLE 9

| wt % | Description/Function | Chemical/Trade Name |
|---|---|---|
| 25% | sanitizing agent/deodorant | triethylene glycol (TEG) |
| 74.85% | propellant/solvent | dimethyl ether (DME) |
| 0.15% | mixture of fragrances | TAKASAGO ™ RK 1428; IFF |
| | | 1401 HBA; Firmenich SJ |
| | | 446138; or similar |
| 100% | | |

### EXAMPLE 10

| wt % | Description/Function | Chemical/Trade Name |
|---|---|---|
| 75% | sanitizing agent/deodorant | triethylene glycol (TEG) |
| 24.85% | propellant/solvent | dimethyl ether (DME) |
| 0.15% | mixture of fragrances | TAKASAGO ™ RK 1428; IFF |
| | | 1401 HBA; Firmenich SJ |
| | | 446138; or similar |
| 100% | | |

Examples 8-10 are single-phase, water-free and ethanol-free formulations that can be dispensed from a metal can without the need for corrosion inhibitors.

### EXAMPLE 11

| wt % | Description/Function | Chemical/Trade Name |
|---|---|---|
| 20% | sanitizing agent/deodorant | triethylene glycol (TEG) |
| 30% | propellant/solvent | dimethyl ether (DME) |
| 40% | solvent | ethanol |
| 10% | B-52 hydrocarbon | butane/propane mixture |
| | propellant mixture | |
| 100% | | |

Example 11 exhibits a single-phase presence when pressurized and observed in a glass aerosol bottle. Moreover, no apparent corrosion is observed when Example 11 is pressurized in a 70mm aluminum can.

### EXAMPLE 12

| wt % | Description/Function | Chemical/Trade Name |
|---|---|---|
| 20% | sanitizing agent/deodorant | triethylene glycol (TEG) |
| 20% | propellant/solvent | dimethyl ether (DME) |
| 40% | solvent | ethanol |
| 20% | B-52 hydrocarbon | butane/propane mixture |
| | propellant mixture | |
| 100% | | |

Example 12 exhibits a single-phase presence when pressurized and observed in a glass aerosol bottle. Moreover, no apparent corrosion is observed when Examples 12 is pressurized in a 70 mm aluminum can.

### EXAMPLE 13

| wt % | Description | Chemical/Trade Name |
|---|---|---|
| 31.47% | sanitizing agent/deodorant | triethylene glycol (TEG) |
| 50% | propellant/solvent | dimethyl ether (DME) |
| 14.93% | solvent | ethanol |
| 3.6% | mixture of fragrances | TAKASAGO ™ RK 1428; IFF |
| | | 1401 HBA; Firmenich SJ |
| | | 446138; or similar |
| 100.00% | | |

Example 13 exhibits a single-phase presence when pressurized and observed in a glass aerosol bottle. Moreover, no apparent corrosion is observed when Example 13 is pressurized in a 70 mm aluminum can.

### EXAMPLE 14

| wt % | Description | Chemical/Trade Name |
|---|---|---|
| 31.47% | sanitizing agent/deodorant | triethylene glycol (TEG) |
| 64.93% | propellant/solvent | dimethyl ether (DME) |
| 0% | solvent | ethanol |
| 0% | solvent | water |
| 3.6% | mixture of fragrances | TAKASAGO ™ RK 1428; IFF |
| | | 1401 HBA; Firmenich SJ |
| | | 446138; or similar |
| 100.00% | | |

### EXAMPLE 15

| wt % | Description | Chemical/Trade Name |
|---|---|---|
| 25% | sanitizing agent/ | triethylene glycol (TEG) |
| | deodorant | |
| 72% | propellant/solvent | dimethyl ether (DME) |
| 0% | solvent | ethanol |
| 0% | solvent | water |
| 3% | mixture of | TAKASAGO ™ RK 1428; IFF |
| | fragrances | 1401 HBA; Firmenich SJ |
| | | 446138; or similar |
| 100.00% | | |

### EXAMPLE 16

| wt % | Description | Chemical/Trade Name |
|---|---|---|
| 35% | sanitizing agent/ | triethylene glycol (TEG) |
| | deodorant | |
| 60% | propellant/solvent | dimethyl ether (DME) |
| 0% | solvent | ethanol |
| 0% | solvent | water |
| 4% | mixture of | TAKASAGO ™ RK 1428; IFF |
| | fragrances | 1401 HBA; Firmenich SJ |
| | | 446138; or similar |
| 100.00% | | |

Like Examples 8-10, Examples 14-16 are single-phase formulations that do not leave substantial amounts of visible residue on a surface after being dispensed in the air of an enclosed room above such a surface. Of course, if excessive amounts of formulation are used, the above formulations can leave some visible residue on a hard surface. However, when used in appropriate amounts or when dispensed automatically with a typical 60 μl metered automatic dispenser, a residue, if present, will not be noticeable to the average consumer. As Examples 8-10 and 14-16 are water-free and alcohol-free, these formulations can be dispensed from metal cans without the need for including corrosion inhibitors.

Thus, the above formulations can be packaged and sold in conventional aerosol dispensers in conventional automated metered aerosol dispensers. Such dispensing devices are well known to those skilled in the art.

While only certain embodiments have been set forth, alternative embodiments and various modifications will be apparent from the above description to those skilled in the art. These and other alternatives are considered equivalents and within the spirit and scope of this disclosure and the appended claims.

What is claimed is:

1. An aerosol formulation comprising:
no less than about 15 wt % glycol; and
an ether propellant,
wherein the aerosol formulation is a single-phase formulation at room temperature.

2. The aerosol formulation of claim 1 wherein the glycol is selected from the group consisting of triethylene glycol (TEG), dipropylene glycol, propylene glycol, and mixtures thereof.

3. The aerosol formulation of claim 1 wherein the ether propellant is selected from the group consisting of dimethyl ether, halogen-substituted dimethyl ether, halogen-substituted methyl ethyl ether, and mixtures thereof.

4. The aerosol formulation of claim 1 wherein the ether propellant is dimethyl ether.

5. The aerosol formulation of claim 1
further comprising a co-solvent selected from the group consisting of water, monohydric short chain alcohols and mixtures thereof.

6. The aerosol formulation of claim 1 further comprising a co-propellant.

7. The aerosol formulation of claim 1 further comprising from about 0.01 to about 6 wt % fragrance.

8. The aerosol formulation of claim 1 wherein the glycol is triethylene glycol (TEG), the ether propellant is dimethyl ether (DME) and the formulation further comprises fragrance.

9. The aerosol formulation of claim 8 wherein the formulation consists essentially of TEG, DME and fragrance.

10. The aerosol formulation of claim 8 wherein TEG is present in an amount ranging from about 25 to about 35 wt %, DME is present in an amount ranging from about 60 to about 70 wt % and fragrance is present in an amount less than 5 wt %.

11. An aerosol formulation comprising:
no less than about 15 wt % glycol; and
from about 10 to about 85 wt % ether propellant,
wherein the aerosol formulation is a single-phase formulation at room temperature.

12. The aerosol formulation of claim 11 wherein the glycol is selected from the group consisting of triethylene glycol (TEG), dipropylene glycol, propylene glycol, and mixtures thereof.

13. The aerosol formulation of claim 12 wherein the ether propellant is selected from the group consisting of dimethyl ether, halogen-substituted dimethyl ether, halogen-substituted methyl ethyl ether, and mixtures thereof.

14. The aerosol formulation of claim 13 wherein the ether propellant is dimethyl ether.

15. The aerosol formulation of claim 11 further comprising a co-solvent selected from the group consisting of water, monohydric short chain alcohols and mixtures thereof.

16. The aerosol formulation of claim 11 further comprising a co-propellant.

17. The aerosol formulation of claim 11 further comprising from about 0.01 to about 5 wt % fragrance.

18. An aerosol formulation comprising:
no less than about 25 wt % triethylene glycol (TEG);
from about 40 to about 85 wt % dimethyl ether (DME) propellant; and
optionally, from about 0.01 to about 6 wt % fragrance,
wherein the aerosol formulation is a single-phase formulation at room temperature.

19. The aerosol formulation of claim 18 wherein the formulation consists essentially of TEG, DME and fragrance.

20. The aerosol formulation of claim 18 wherein TEG is present in an amount ranging from about 25 to about 35 wt %, DME is present in an amount ranging from about 60 to about 70 wt % and fragrance is present in an amount less than 6 wt %.
